(19) United States
(12) Patent Application Publication
(10) Pub. No.: US 2024/0423883 A1
(43) Pub. Date: Dec. 26, 2024

(54) COMPOSITION FOR LIGHTENING KERATIN FIBRES, COMPRISING A PEROXYGENATED SALT, CARBONATE AND/OR AMMONIUM BICARBONATE AND AT LEAST 1% OF A POLYPHOSPHORUS DERIVATIVE

(71) Applicant: L'Oreal, Paris (FR)

(72) Inventors: Fabien Aubert, Saint Ouen (FR); Karine Lucet-Levannier, Saint Ouen (FR); Alexis Liard, Saint Ouen (FR); Ambre Souvirou, Saint Ouen (FR)

(73) Assignee: L'Oreal, Paris (FR)

(21) Appl. No.: 18/292,286

(22) PCT Filed: Jul. 26, 2022
(86) PCT No.: PCT/EP22/70993

§ 371 Date: Jan. 25, 2024

(30) Foreign Application Priority Data
Jul. 27, 2021 (FR) 2108161

(51) Int. Cl. A61K 8/22; A61K 8/24; A61K 8/55; A61K 8/73; A61Q 5/08

(52) U.S. Cl. CPC A61K 8/22; A61K 8/24; A61K 8/55; A61K 8/731; A61Q 5/08; A61K 2800/882

(57) ABSTRACT

The present invention relates to a composition for lightening keratin fibres, preferably human keratin fibres such as the hair, comprising: a) one or more peroxygenated salt(s); b) at least one agent chosen from ammonium carbonate, ammonium bicarbonate and a mixture thereof; and c) at least 1% by weight of one or more polyphosphorus derivative(s) relative to the total weight of the composition, said polyphosphorus derivative(s) being different from said peroxygenated salt(s).

The present invention relates to a composition for lightening keratin fibres, preferably human keratin fibres such as the hair, comprising one or more peroxygenated salt(s), at least one agent chosen from ammonium carbonate, ammonium bicarbonate and a mixture thereof, and at least 1% by weight of one or more polyphosphorus derivative(s) relative to the total weight of the composition.

A subject of the present invention is also a process for lightening keratin fibres, comprising the application of said composition to said keratin fibres.

The present invention also relates to a particular device comprising at least two compartments, which is suitable for using said lightening composition.

### TECHNICAL FIELD

The present invention relates to the field of lightening keratin fibres and more particularly to that of lightening the hair.

The lightening of head hair is evaluated by the “tone depth”, which characterizes the degree or level of lightening. The notion of “tone” is based on the classification of natural shades, one tone separating each shade from the shade immediately following or preceding it. This definition and the classification of natural shades are well known to hairstyling professionals and are published in the book Sciences des traitements capillaires [The Science of Hair Care] by Charles Zviak, 1988, published by Masson, pp. 215 and 278.

Tone depths range from 1 (black) to 10 (very light blond), one unit corresponding to one tone; the higher the figure, the lighter the shade.

Lightening makes it possible to afford a lighter tone depth than the initial natural tone depth of the head of hair, which is particularly sought by consumers.

The processes for lightening human keratin fibres that are usually employed consist in using an aqueous composition comprising at least one oxidizing agent, under alkaline pH conditions in the vast majority of cases. This oxidizing agent has the role of degrading the melanin of the hair, which, depending on the nature of the oxidizing agent present, leads to more or less pronounced lightening of the fibres. Thus, for relatively mild lightening, the oxidizing agent is generally hydrogen peroxide. When more pronounced lightening is desired, use is usually made of peroxygenated salts, for instance persulfates, in the presence of hydrogen peroxide.

The lightening process is generally carried out under very alkaline conditions. The alkaline agent most commonly used is aqueous ammonia. The use of aqueous ammonia is particularly advantageous in processes of this type. Specifically, it makes it possible to adjust the pH of the composition to an alkaline pH to enable activation of the oxidizing agent. This alkaline agent also causes swelling of the keratin fibre, with opening of the scales, which promotes the penetration of the oxidizing agent into the fibre, and thus increases the efficacy of the reaction.

It has also been proposed to replace all or some of the aqueous ammonia with one or more other conventional basifying agents, such as alkanolamines. However, the solutions proposed hitherto do not result in compositions that are as effective as those based on aqueous ammonia, especially since these basifying agents do not provide sufficient lightening of the pigmented fibres in the presence of the oxidizing agent. Furthermore, monoethanolamine, when used at high concentrations, may cause irritation of the scalp.

Thus, the hair-lightening technique, which must make it possible to obtain sufficient lightening of the fibre, generally involves using either aqueous ammonia or monoethanolamine, or a mixture of monoethanolamine and aqueous ammonia, as basifying agent as a mixture with aqueous hydrogen peroxide solution.

However, these conventional bleaching operations often cause a relatively unaesthetic orangey-yellow glint.

Moreover, systems involving the use of particular alkali metal carbonates, such as potassium carbonate, resulting in marked levels of lightening, have also been proposed.

However, compositions which are even more effective are sought.

Thus, one of the objectives of the present invention is to propose compositions for lightening keratin fibres, preferably human keratin fibres such as the hair, which do not have the drawbacks mentioned above, and which are capable of providing very good lightening performance qualities, with an aesthetic glint, while at the same time having working qualities that are superior to those of the existing compositions.

In particular, the invention makes it possible to obtain good levels of lightening (strength of the lightening obtained) with compositions comprising low contents of peroxygenated salts.

### DISCLOSURE OF THE INVENTION

A subject of the present invention is thus a composition (A) for lightening keratin fibres, preferably human keratin fibres such as the hair, comprising:
a) one or more peroxygenated salts;
b) at least one agent chosen from ammonium carbonate, ammonium bicarbonate and a mixture thereof; and
c) at least 1% by weight of one or more polyphosphorus derivative(s) relative to the total weight of the composition, said polyphosphorus derivative(s) preferably being chosen from linear or cyclic compounds comprising at least two phosphorus atoms bonded together covalently via at least one linker L comprising at least one oxygen atom and/or at least one carbon atom,
said polyphosphorus derivative(s) being different from said peroxygenated salt(s).

For the purposes of the present invention, the expression “a composition for lightening keratin fibres” denotes a composition which is applied to keratin fibres, preferably human keratin fibres such as the hair, in order to lighten them. The composition for lightening keratin fibres according to the invention may be a ready-to-use composition or else a composition that is mixed, just before use, with another composition.

In the remainder of the text, the expressions “lightening composition” and “composition for lightening” are equivalent.

Another subject of the present invention is a process for lightening keratin fibres, in particular human keratin fibres such as the hair.

The invention also relates to a device containing at least two compartments for using the composition according to the invention.

The compositions according to the invention can thus give very good lightening performance qualities on keratin fibres, notably in terms of lightening level and uniformity. These compositions make it possible to obtain lightening of keratin fibres with a visually more natural, more aesthetic appearance, notably without an orangey-yellow glint.

Other features and advantages of the invention will emerge more clearly on reading the description and the examples that follow.

In the text hereinbelow, and unless otherwise indicated, the limits of a range of values are included within that range.

The human keratin fibres treated via the process according to the invention are preferably the hair. The expression “at least one” is equivalent to the expression “one or more”.

### Peroxygenated Salt

The lightening composition (A) according to the invention comprises a) one or more peroxygenated salt(s).

The peroxygenated salt(s) is (are) different from the polyphosphorus derivative(s) described below.

Advantageously, the peroxygenated salt(s) is/are chosen from persulfates, perborates, magnesium peroxide, and mixtures thereof, preferably from persulfates, perborates of alkali metals, such as potassium or sodium, magnesium peroxide and mixtures thereof, more preferentially from the persulfates, even more preferentially from alkali metal persulfates, calcium persulfates, ammonium persulfates and mixtures thereof, better still from potassium persulfates, ammonium persulfates and mixtures thereof.

Preferably, the composition (A) according to the invention comprises at least one persulfate as peroxygenated salt, and more preferably at least one potassium and/or ammonium persulfate.

Particularly preferably, the composition (A) according to the invention comprises at least two persulfates, in particular at least one potassium persulfate and at least one ammonium persulfate.

Advantageously, the total content of peroxygenated salt(s) ranges from 0.1% to 50% by weight, preferably from 1% to 40% by weight, more preferentially from 5% to 30% and better still from 10% to 25% by weight relative to the total weight of the composition (A).

### Ammonium (Bi)Carbonate

The lightening composition (A) according to the invention also comprises at least b) one agent chosen from ammonium carbonate, ammonium bicarbonate and a mixture thereof.

The lightening composition (A) can thus comprise ammonium carbonate or ammonium bicarbonate, or ammonium carbonate and ammonium bicarbonate.

Preferably, the lightening composition (A) does not comprise sodium (bi)carbonate.

Advantageously, the lightening composition (A) does not comprise other (bi)carbonate(s) than ammonium carbonate, ammonium bicarbonate and their mixture. In other terms, according to this embodiment, the agent b) chosen from ammonium carbonate, ammonium bicarbonate and a mixture thereof, may be the only (bi)carbonate(s) present in the lightening composition (A).

Advantageously, the total content of agent(s) b) ranges from 1% to 40% by weight, preferably from 5% to 40% by weight, more preferentially from 10% to 20% by weight, relative to the total weight of the composition.

### Polyphosphorus Derivative

The lightening composition (A) according to the invention also comprises at least 1% by weight of one or more polyphosphorus derivative(s) relative to the total weight of the composition, said polyphosphorus derivative(s) being different from said peroxygenated salt(s) described above.

The term “polyphosphorus derivative” means a compound comprising at least 2 phosphorus atoms.

Preferably, said polyphosphorus derivative(s) are chosen from linear or cyclic compounds comprising at least two phosphorus atoms bonded together covalently via at least one linker L comprising at least one oxygen atom and/or at least one carbon atom.

According to one embodiment, when the linker comprises at least one carbon atom, it may comprise at least one nitrogen atom.

Preferably, the polyphosphorus derivative(s) used according to the present invention comprises (comprise) less than 20 phosphorus atoms, more preferentially less than 15 phosphorus atoms, even more preferentially less than 10 phosphorus atoms.

Advantageously, said polyphosphorus derivative(s) comprises (comprise) at least two groups chosen from a group —P(R)(═O)—OH, a group —P(R)(═O)—OM, a group >P(═O)—OH and a group >P(═O)—OM, with:
M representing a cationic counterion, preferably chosen from alkali metals and alkaline-earth metals,
R representing a hydroxyl group, a group —OM′, M′ representing a cationic counterion, preferably chosen from alkali metals and alkaline-earth metals, a (C₁-C₆)alkyl group, a (C₁-C₆)alkoxy group, a cycloalkyloxy group or a (hetero)aryloxy group, and
> representing the two bonds connected to the phosphorus atom and forming part of a ring.

According to a preferred embodiment, the polyphosphorus derivative(s) are chosen from inorganic polyphosphorus derivatives.

According to another embodiment, the polyphosphorus derivative(s) are chosen from organic polyphosphorus derivatives.

Preferably, the polyphosphorus derivative(s) present in the compositions according to the invention is/are non-amine derivative(s).

Preferably, the polyphosphorus derivative(s) is/are chosen from polyphosphates and polyphosphonates, and mixtures thereof.

Preferably, the polyphosphorus derivative(s) are polyphosphates.

Advantageously, said polyphosphorus derivative(s) is/are chosen from compounds belonging to any one of formulae (I), (II) and (III) below, or mixtures thereof:

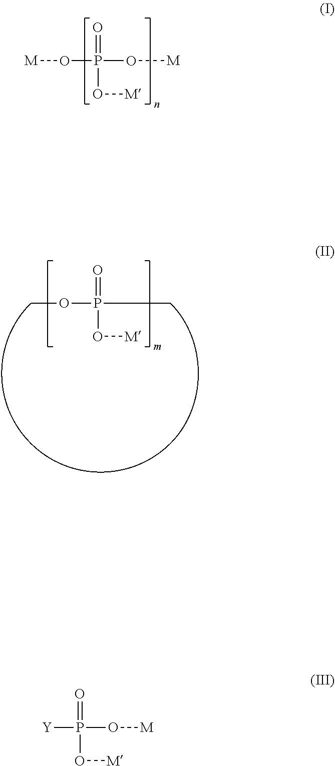

and also the solvates thereof such as hydrates,
in which formulae:
n ranges from 2 to 10, preferably from 2 to 6 and more preferentially from 2 to 3;
m ranges from 2 to 10, preferably from 2 to 6;
Y representing an alkyl chain comprising at least one phosphorus atom and optionally one or more non-phosphorus heteroatoms, or a cyclic carbon-based radical optionally comprising one or more heteroatoms, said hydrocarbon-based radical being substituted with one or more groups comprising one or more phosphorus atoms;
M or M′ representing, independently one another, a hydrogen atom, an alkali metal or an alkaline-earth metal;
---- representing a single bond when M or M′ is H, or an ionic bond.

It is understood that when M or M′ is other than H, then M or M′ are such that the overall charge of the molecule is zero. Thus, in the case of divalent metals, M and M′ may represent the same divalent metal.

The polyphosphorus derivatives of formula (I) are linear.

The polyphosphorus derivatives of formula (II) are cyclic.

Advantageously, the polyphosphorus derivative(s) is/are chosen from:
inorganic polyphosphorus derivatives chosen from:
pyrophosphates, preferably in the form of salts, preferably of alkali metal salts, which may or may not be hydrated, such as sodium pyrophosphate, potassium pyrophosphate or sodium pyrophosphate decahydrate;
hexametaphosphates, preferably in the form of salts, preferably of alkali metal salts, which may or may not be hydrated, such as sodium hexametaphosphate;
tripolyphosphates, preferably in the form of salts, preferably of alkali metal salts, which may or may not be hydrated, such as sodium tripolyphosphate;
trimetaphosphates, preferably in the form of salts, preferably of alkali metal salts, which may or may not be hydrated, such as sodium trimetaphosphate;
and mixtures thereof,
and organic polyphosphorus derivatives, preferably chosen from:
organic polyphosphate derivatives, such as polyphosphoric acids and/or salts thereof such as phytic acid (also known as myoinositol hexaphosphoric acid);
organic polyphosphonate derivatives, such as polyphosphonic acids and/or salts thereof such as ethylenediaminetetramethylene phosphonic acid (EDTMP), diethylenetriaminepentamethylene phosphonic acid (DETMP), aminotrimethylene phosphonic acid (ATMP), hydroxyethylene diphosphonic acid (HEDP), diethylenetriamine pentamethylene phosphonic acid (DTPMP) and mixtures thereof,
and mixtures thereof.

Advantageously, the polyphosphorus derivative(s) is/are chosen from:
inorganic polyphosphate derivatives chosen from hydrated or non-hydrated alkali metal pyrophosphates, such as sodium pyrophosphate, potassium pyrophosphate, sodium pyrophosphate decahydrate; and polyphosphates, such as sodium hexametaphosphate, sodium polyphosphate, sodium tripolyphosphate, sodium trimetaphosphate; and mixtures thereof;
organic polyphosphorus derivatives chosen from polyphosphoric acids and/or salts thereof such as phytic acid (also known as myoinositol hexaphosphoric acid), polyphosphonic acids and/or salts thereof such as EDTMP, DETMP, ATMP, HEDP, DTPMP, and mixtures thereof,
and mixtures thereof;
   preferably from inorganic polyphosphate derivatives.

Preferably, according to a first embodiment, the polyphosphorus derivative(s) are inorganic polyphosphate compounds, preferably chosen from:
polyphosphates, and/or hydrates thereof; and mixtures thereof, preferably of sodium and/or potassium, such as sodium hexametaphosphate (SHMP),

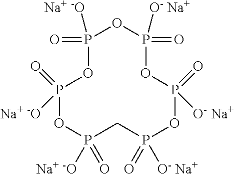

sodium polyphosphate, sodium tripolyphosphate, sodium trimetaphosphate, preferably sodium tripolyphosphate having the following formula:

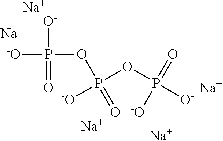

pyrophosphates and/or hydrates thereof, and mixtures thereof, preferably of sodium and/or potassium; preferably chosen from sodium pyrophosphate and/or potassium pyrophosphate, and hydrates thereof, such as
   sodium pyrophosphate decahydrate having the following formula:

4Na⁺·10H₂O

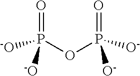

or potassium pyrophosphate having the following formula:

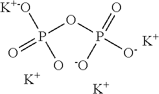

and mixtures thereof.

According to a second embodiment, the polyphosphorus derivative(s) are organic polyphosphate derivatives and/or organic polyphosphonate derivatives, preferably chosen from polyphosphoric acids and/or salts thereof, polyphosphonic acids and/or salts thereof such as EDTMP, DETMP, ATMP, HEDP, DTPMP

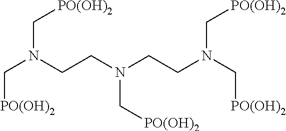

DETMP and mixtures thereof,
iminodi(methylphosphonic) acid (example L) or salts thereof, and mixtures thereof;

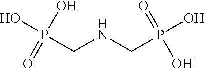 [SMILES: O=P(O)(O)CNCP(=O)(O)O]

tetrasodium etidronate (example K) of formula:

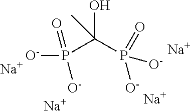

phytic acid of formula:

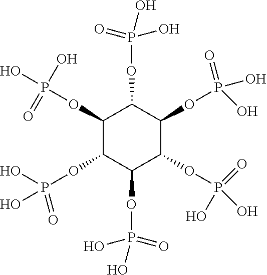 [SMILES: O=P(O)(O)O[C@H]1[C@H](OP(=O)(O)O)[C@@H](OP(=O)(O)O)[C@H](OP(=O)(O)O)[C@@H](OP(=O)(O)O)[C@@H]1OP(=O)(O)O]

and mixtures thereof.

Preferably, the polyphosphorus derivatives are chosen from inorganic polyphosphates, preferably chosen from: optionally hydrated alkali metal pyrophosphates, preferably chosen from sodium pyrophosphate, potassium pyrophosphate, sodium pyrophosphate decahydrate; and polymetaphosphates, such as sodium hexametaphosphate, sodium trimetaphosphate; sodium polyphosphates such as sodium tripolyphosphate, and mixtures thereof. Sodium and/or potassium is preferably used as alkali metal.

According to another embodiment, the organic polyphosphorus derivatives are chosen from polyphosphoric acids and/or salts thereof, such as phytic acid, polyphosphonic acids and/or salts thereof, such as EDTMP, DETMP, ATMP, HEDP, DTPMP, and mixtures thereof.

Advantageously, the polyphosphorus derivative(s) is/are chosen from inorganic polyphosphate derivatives, preferably from hydrated or non-hydrated alkali metal pyrophosphates, more preferentially from sodium pyrophosphate, potassium pyrophosphate and sodium pyrophosphate decahydrate, and mixtures thereof.

As indicated above, said polyphosphorus derivative(s) are different from said peroxygenated salt(s). Particularly preferably, the polyphosphorus derivative(s) are not peroxygenated salts.

Advantageously, the total content of polyphosphorus derivative(s) c) ranges from 1% to 40% by weight, preferably from 2% to 30% by weight, more preferentially from 3% to 20% by weight and better still from 5% to 15% by weight relative to the total weight of the composition (A).

### Thickener

The lightening composition (A) according to the invention may also comprise one or more thickener(s).

Preferably, the thickener(s) may be chosen from mineral thickeners, organic thickeners and mixtures thereof.

In particular, the thickeners may be mineral thickeners chosen from organophilic clays and fumed silicas, or mixtures thereof.

The organophilic clay may be chosen from montmorillonite, bentonite, hectorite, attapulgite, sepiolite and mixtures thereof. The clay is preferably a bentonite or a hectorite.

These clays can be modified with a chemical compound chosen from quaternary amines, tertiary amines, amine acetates, imidazolines, amine soaps, fatty sulfates, alkylarylsulfonates, amine oxides, and mixtures thereof.

Mention may be made, as organophilic clays, of quaternium-18 bentonites, such as those sold under the names Bentone 3, Bentone 38 and Bentone 38V by Rheox, Tixogel VP by United Catalyst and Claytone 34, Claytone 40 and Claytone XL by Southern Clay; stearalkonium bentonites, such as those sold under the names Bentone 27 by Rheox, Tixogel LG by United Catalyst and Claytone AF and Claytone APA by Southern Clay; or quaternium-18/benzalkonium bentonites, such as those sold under the names Claytone HT and Claytone PS by Southern Clay.

The fumed silicas can be obtained by high-temperature pyrolysis of a volatile silicon compound in an oxyhydrogen flame, producing a finely divided silica. This process makes it possible in particular to obtain hydrophilic silicas which exhibit a large number of silanol groups at their surface. Such hydrophilic silicas are sold, for example, under the names Aerosil 130®, Aerosil 200®, Aerosil 255®, Aerosil 300® and Aerosil 380® by Degussa, and Cab-O-Sil HS-5®, Cab-O-Sil EH-5®, Cab-O-Sil LM-130®, Cab-O-Sil MS-55® and Cab-O-Sil M-5® by Cabot.

It is possible to chemically modify the surface of the silica by chemical reaction for the purpose of reducing the number of silanol groups. It is possible in particular to replace silanol groups with hydrophobic groups: a hydrophobic silica is then obtained.

The hydrophobic groups can be:
trimethylsiloxyl groups, which are notably obtained by treating fumed silica in the presence of hexamethyldisilazane. Silicas thus treated are known as “Silica silylate” according to the CTFA (6th Edition, 1995). They are sold, for example, under the references Aerosil R812® by Degussa and Cab-O-Sil TS-530® by Cabot.
dimethylsilyloxyl or polydimethylsiloxane groups, which are obtained in particular by treating fumed silica in the presence of polydimethylsiloxane or dimethyldichlorosilane. Silicas thus treated are known as Silica dimethyl silylate according to the CTFA (6th Edition, 1995). They are sold, for example, under the references Aerosil R972® and Aerosil R974® by Degussa, and Cab-O-Sil TS-610® and Cab-O-Sil TS-720® by Cabot.

The fumed silica preferably exhibits a particle size which can be nanometric to micrometric, for example ranging from approximately 5 to 200 nm.

When it is present, the mineral thickener preferably represents from 0.1% to 20% by weight, more preferentially from 0.1% to 10% by weight relative to the weight of the composition.

As indicated above, the thickener(s) can be chosen from organic thickeners.

These thickeners may be chosen from fatty acid amides (coconut monoethanolamide or diethanolamide, oxyethylenated carboxylic acid alkyl ether monoethanolamide), polymeric thickeners such as cellulose-based thickeners (hydroxyethylcellulose, hydroxypropylcellulose or carboxymethylcellulose), guar gum and derivatives thereof (hydroxypropyl guar), gums of microbial origin (xanthan gum, scleroglucan gum), starches, crosslinked acrylic acid or acrylamidopropanesulfonic acid homopolymers and associative polymers (polymers comprising hydrophilic regions and fatty-chain hydrophobic regions (alkyl or alkenyl containing at least 10 carbon atoms) that are capable, in an aqueous medium, of reversibly combining with each other or with other molecules).

According to one particular embodiment, the thickener(s) are chosen from organic thickeners, preferably chosen from cellulose-based thickeners (hydroxyethylcellulose, hydroxypropylcellulose or carboxymethylcellulose), guar gum and derivatives thereof (hydroxypropyl guar), gums of microbial origin (xanthan gum or scleroglucan gum) and associative polymers, and more preferentially from cellulose-based thickeners in particular with hydroxyethylcellulose.

When it (they) is (are) present, the total content of thickener(s) can range from 0.1% to 20% by weight, preferably from 0.1% to 10% by weight, relative to the weight of the composition.

When it (they) is (are) present, the total content of organic thickener(s) can range from 0.1% to 20% by weight, preferably from 0.1% to 10% by weight, relative to the total weight of the lightening composition according to the invention.

The lightening composition (A) according to the invention may comprise one or more cyclodextrin(s).

When it is present, the total content of cyclodextrin(s) ranges from 5% to 80% by weight, preferably from 10% to 60% by weight, more preferentially from 20% to 60% by weight, relative to the total weight of the composition.

The lightening composition (A) according to the invention may be aqueous.

According to one particular embodiment, the lightening composition (A) according to the invention is anhydrous.

For the purposes of the present invention, the term “anhydrous composition” is intended to mean a composition having a water content of less than 2% by weight, preferably less than 1% by weight, relative to the weight of the composition.

According to one particular embodiment, the lightening composition (A) according to the invention does not comprise any organic amines such as alkanolamines and in particular monoethanolamine.

According to one particular embodiment, the lightening composition (A) according to the invention does not comprise aqueous ammonia.

Particularly preferably, the lightening composition (A) according to the invention does not comprise any organic amines, such as alkanolamines and in particular monoethanolamine, nor any aqueous ammonia.

### Fatty Substance

The lightening composition (A) according to the invention may also comprise one or more fatty substances.

For the purposes of the present invention, the term “fatty substance” means an organic compound that is insoluble in water at ordinary ambient temperature (20-25° C.) and at atmospheric pressure (760 mmHg, i.e. 1.013×10⁵ Pa), with a solubility in water of less than 5%, preferably less than 1% and even more preferentially less than 0.1%. The fatty substances generally have in their structure a hydrocarbon-based chain comprising at least 6 carbon atoms. In addition, the fatty substances are generally soluble in organic solvents under the same temperature and pressure conditions, for instance chloroform, ethanol, benzene, liquid petroleum jelly or decamethylcyclopentasiloxane.

The fatty substances are, moreover, non-(poly)oxyalkylenated and non-(poly)glycerolated. In other words, the fatty substances do not comprise in their structure a (poly)ethylene oxide or (poly)glycerol or (poly)propylene glycol unit.

The fatty substance(s) may be chosen from solid fatty substances and/or liquid fatty substances (also called “oil”), and mixtures thereof.

The term “oil” means a “fatty substance” which is liquid, i.e. which is capable of flowing under the action of its own weight at ambient temperature (25° C.) and at atmospheric pressure (760 mmHg, i.e. 1.013×10⁵ Pa). Preferably, the viscosity at a temperature of 25° C. and at a shear rate of 1 s⁻¹ of the oil is between 10⁻³ Pa·s and 2 Pa·s. It may be measured using a Thermo Haake RS600 rheometer with cone-plate geometry or an equivalent machine.

For the purposes of the present invention, the term “solid fatty substance” means a fatty substance that is not liquid at ambient temperature (20-25° C.) and at atmospheric pressure (760 mmHg, i.e. 1.013×10⁵ Pa), in particular a solid compound or a compound having a viscosity of greater than 2 Pa·s at a shear rate of 1 s⁻¹ under the conditions mentioned above.

Preferably, the fatty substance(s) are chosen from the liquid fatty substances.

Advantageously, the liquid fatty substances are chosen from:
fatty alcohols that are liquid at ambient temperature, bearing a branched and/or unsaturated carbon-based chain containing from 12 to 26 carbon atoms, for instance cetanol, octyldodecanol, stearyl alcohol, cetearyl alcohol, linoleyl alcohol, linolenyl alcohol, isostearyl alcohol, oleyl alcohol, 2-hexyldecanol, 2-butyloctanol or 2-undecylpentadecanol;
C₆-C₁₆ hydrocarbons;
hydrocarbons comprising more than 16 carbon atoms;
silicones and
   mixtures thereof.

Advantageously, the total content of fatty substances, when they are present, ranges from 0.1% to 10% by weight, preferably from 1% to 10% by weight, relative to the total weight of the lightening composition according to the invention.

### Direct Dyes

The lightening composition (A) according to the invention can also comprise one or more synthetic or natural direct dye(s).

They may be ionic or non-ionic, preferably cationic or non-ionic.

Examples of suitable direct dyes that may be mentioned include azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

The direct dyes are preferably cationic direct dyes. Mention may be made of the hydrazono cationic dyes of formulae (III) and (IV) and the azo cationic dyes (V) and (VI) below:

Het⁺-N(Ra)—N═C(Rb)—Ar,Q- (III),

Het⁺-C(Ra)=N—C(Rb)—Ar,Q- (IV),

Het⁺-N═N—Ar,Q- (V),

Ar⁺—N═N—Ar″,Q- (VI),
in which formulae (III) to (VI):
   Het⁺ represents a cationic heteroaryl radical, preferentially bearing an endocyclic cationic charge, such as imidazolium, indolium or pyridinium, which is optionally substituted, preferentially with at least one (C₁-C₈)alkyl group such as methyl;
   Ar⁺ represents an aryl radical, such as phenyl or naphthyl, bearing an exocyclic cationic charge, preferentially ammonium, particularly tri(C₁-C₈)alkylammonium, such as trimethylammonium;
   Ar represents an aryl group, notably phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
   Ar″ represents an optionally substituted (hetero)aryl group, such as phenyl or pyrazolyl, which are optionally substituted, preferentially with one or more (C₁-C₈)alkyl, hydroxyl, (di)(C₁-C₈)(alkyl)amino, (C₁-C₈)alkoxy or phenyl groups;
   Ra and Rb, which may be identical or different, represent a hydrogen atom or a (C₁-C₈)alkyl group, which is optionally substituted, preferentially with a hydroxyl group;
or else the substituent Ra with a substituent of Het⁺ and/or Rb with a substituent of Ar form, together with the atoms that bear them, a (hetero)cycloalkyl; in particular, Ra and Rb represent a hydrogen atom or a (C₁-C₄)alkyl group optionally substituted with a hydroxyl group;
   Q⁻ represents an organic or mineral anionic counterion, such as a halide or an alkyl sulfate.

In particular, mention may be made of the azo and hydrazono direct dyes bearing an endocyclic cationic charge of formulae (III) to (VI) as defined previously, more particularly, the cationic direct dyes bearing an endocyclic cationic charge described in patent applications WO 95/15144, WO 95/01772 and EP 714 954, preferentially the following direct dyes:

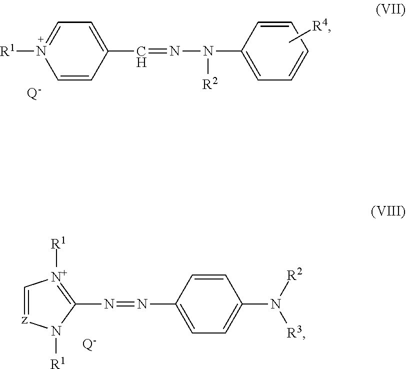

in which formulae (VII) and (VIII):
   R¹ represents a (C₁-C₄)alkyl group such as methyl;
   R² and R³, which may be identical or different, represent a hydrogen atom or a (C₁-C₄)alkyl group, such as methyl; and
   R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; in particular, R⁴ is a hydrogen atom;
   Z represents a CH group or a nitrogen atom, preferentially CH,
   Q− is an anionic counterion as defined previously, in particular a halide, such as chloride, or an alkyl sulfate, such as methyl sulfate or mesyl.

In particular, the dyes of formulae (V) and (VI) are chosen from Basic Red 51, Basic Yellow 87 and Basic Orange 31 or derivatives thereof with Q′ being an anionic counterion as defined previously, particularly a halide such as chloride, or an alkyl sulfate such as methyl sulfate or mesyl.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin, apigenidin and orceins. Use may also be made of extracts or decoctions containing these natural dyes and notably henna-based poultices or extracts.

When they are present, the content of direct dye(s) may range from 0.001% to 10% by weight, preferably from 0.005% to 5% by weight, relative to the total weight of the composition (A).

### Additives

The lightening composition (A) according to the invention may contain various adjuvants conventionally used in compositions for lightening keratin fibres, such as the hair, such as anionic, cationic, non-ionic, amphoteric or zwitterionic polymers or mixtures thereof which are different from the thickeners described above; antioxidants; penetrants; sequestrants; fragrances; dispersants; film-forming agents; ceramides; preserving agents; opacifiers.

When they are present, the content of adjuvants above can range for each of them from 0.01% to 40% by weight relative to the weight of the composition, and preferably from 0.1% to 20% by weight relative to the weight of the composition.

### Process for Lightening Keratin Fibres

The present invention also relates to a process for lightening keratin fibres, preferably human keratin fibres such as the hair, comprising the application to the hair of at least one lightening composition (A) according to the invention.

Preferably, the process according to the invention comprises a step of mixing the lightening composition (A) according to the invention with an oxidizing composition (B) comprising at least one chemical oxidizing agent, followed by the application to the keratin fibres of the composition resulting from the mixing of the compositions (A) and (B).

This mixing step is preferably performed at the time of use, just before applying to the hair the composition resulting from the mixing.

Preferably, the chemical oxidizing agent is hydrogen peroxide.

The oxidizing composition (B) is preferably an aqueous composition. In particular, it comprises more than 5% by weight of water, preferably more than 10% by weight of water, and even more advantageously more than 20% by weight of water, relative to the total weight of the oxidizing composition.

It may also comprise one or more organic solvents.

By way of organic solvent, mention may for example be made of linear or branched C₂ to C₄ alkanols, such as ethanol and isopropanol; glycerol; polyols and polyol ethers such as 2-butoxyethanol, propylene glycol, dipropylene glycol, propylene glycol monomethyl ether, and diethylene glycol monoethyl ether and monomethyl ether, and also aromatic alcohols or ethers, such as benzyl alcohol or phenoxyethanol, and mixtures thereof.

The organic solvent(s) may be present in an amount ranging from 1% to 40% by weight, and preferably ranging from 5% to 30% by weight, relative to the weight of the oxidizing composition.

The oxidizing composition may also preferably comprise one or more acidifying agents. Among the acidifying agents, examples that may be mentioned include mineral or organic acids, for instance hydrochloric acid, orthophosphoric acid, sulfuric acid, carboxylic acids, for instance acetic acid, tartaric acid, citric acid or lactic acid, and sulfonic acids.

The oxidizing composition may also comprise fatty substances such as those described above, preferably chosen from fatty alcohols, liquid hydrocarbons comprising more than 16 carbon atoms and mixtures thereof, surfactants and polymers.

Usually, the pH of the oxidizing composition, when it is aqueous, is less than 7.

According to one particular embodiment, the composition resulting from the mixing of the compositions (A) and (B) is such that the total content of c) polyphosphorus derivative(s) is at least 1% by weight relative to the weight of the composition resulting from the mixing of the compositions (A) and (B).

Advantageously, the chemical oxidizing agent is hydrogen peroxide.

Preferably, the oxidizing composition comprises hydrogen peroxide as oxidizing agent, in aqueous solution, the concentration of which can range from 0.1% to 50%, more preferentially between 0.5% and 20% and even more preferentially between 1% and 15% by weight, relative to the weight of the oxidizing composition.

The composition applied to the hair can result from the mixing of at least two compositions, in particular of two compositions:
a composition (A) comprising:
a) one or more peroxygenated salt(s), b) at least one agent chosen from ammonium carbonate, ammonium bicarbonate and a mixture thereof, and c) at least one polyphosphorus derivative(s) as defined above, the total content of c) polyphosphorus derivative(s) being at least 1% by weight relative to the weight of the composition (A), and
a composition (B) comprising at least one chemical oxidizing agent, preferably hydrogen peroxide.

This composition is a ready-to-use composition.

Advantageously, the ready-to-use composition applied to the hair results from the mixing of at least two compositions, in particular of two compositions:
a composition (A) comprising:
a) one or more peroxygenated salt(s), b) at least one agent chosen from ammonium carbonate, ammonium bicarbonate and a mixture thereof, and c) at least one polyphosphorus derivative(s) as defined above; and
a composition (B) comprising at least one chemical oxidizing agent, preferably hydrogen peroxide,
   such that the total content of c) polyphosphorus derivative(s) is at least 1% by weight relative to the weight of the composition resulting from the mixing of the compositions (A) and (B).

Preferably, the composition resulting from the mixing of the compositions (A) and (B), namely the ready-to-use composition, has a pH of between 8 and 10, preferably between 8.5 and 9.5, more preferably equal to 8.9.

The composition may be applied to wet or dry keratin fibres. On conclusion of the treatment, the keratin fibres are optionally rinsed with water, optionally washed with a shampoo and then rinsed with water, before being dried or left to dry.

The composition is left in place for a time generally from 1 minute to 1 hour, preferably from 5 minutes to 50 minutes.

The temperature during the lightening process is conventionally between ambient temperature (between 15° C. and 25° C.) and 80° C., and preferably between ambient temperature and 60° C.

On conclusion of the treatment, the keratin fibres, preferably human keratin fibres, are optionally rinsed with water, optionally washed with a shampoo and then rinsed with water, before being dried or left to dry.

Preferably, at least one of the compositions (A) or (B) is aqueous.

Even more preferentially, only the composition (B) is aqueous.

The compositions (A) and (B) are preferably mixed in a weight ratio (A)/(B) ranging from 0.2 to 10 and better still from 0.5 to 2.

A subject of the invention is also a ready-to-use composition comprising a) one or more peroxygenated salt(s), b) at least one agent chosen from ammonium carbonate, ammonium bicarbonate and a mixture thereof, and c) at least one polyphosphorus derivative(s) as defined above, the total content of c) polyphosphorus derivative(s) being at least 1% by weight relative to the weight of the ready-to-use composition and d) hydrogen peroxide.

### Device

The present invention also relates to a device comprising at least two compartments containing:
in a first compartment, a lightening composition (A) according to the invention;
in a second compartment, a composition (B) comprising at least one chemical oxidizing agent, preferably hydrogen peroxide,
   the compositions (A) and (B) being intended to be mixed before application in order to obtain the composition resulting from the mixing of the compositions (A) and (B). According to one embodiment, the content of c) polyphosphorus derivative(s) is at least 1% by weight relative to the weight of the composition resulting from the mixing of the compositions (A) and (B).

Advantageously, the device comprises at least two compartments containing:
in a first compartment, a composition (A) comprising a) one or more peroxygenated salt(s), b) at least one agent chosen from ammonium carbonate, ammonium bicarbonate and a mixture thereof, and c) at least one polyphosphorus derivative(s) as defined above;
in a second compartment, a composition (B) comprising hydrogen peroxide,
   the compositions (A) and (B) being intended to be mixed before application in order to obtain the composition resulting from the mixing of the compositions (A) and (B). According to one embodiment, the content of c) polyphosphorus derivative(s) is at least 1% by weight relative to the weight of the composition resulting from the mixing of the compositions (A) and (B).

The present invention will now be described more specifically by means of examples, which do not in any way limit the scope of the invention. However, the examples make it possible to support specific characteristics, variants and preferred embodiments of the invention.

In these examples, the colour of the locks was evaluated in the CIE L*a*b* system, using a Minolta Spectrophotometer CM3600A colorimeter (illuminant D65, angle 10°, specular component included).

In this L*a*b* system, L* represents the intensity of the colour, a* indicates the green/red colour axis and b* the blue/yellow colour axis. The higher the value of L*, the lighter the colour. The higher the value of a*, the redder the colour and the higher the value of b*, the yellower the colour.

The variation or extent of the lightening between untreated locks of hair and locks of hair after treatment is defined by the parameter ΔE and is calculated according to the following equation:

ΔE=\sqrt{(L*−L₀*)²+(a*−a₀*)²+(b*−b₀*)²}

In this equation, the parameters L*, a* and b* represent the values measured on locks of hair after lightening and the parameters L₀*, a₀* and b₀* represent the values measured on untreated locks of hair. The higher the value of ΔE, the better the lightening of the keratin fibres.

### EXAMPLES

### Example 1

The compositions A1 and A2 as described below were prepared: unless otherwise mentioned, the amounts are expressed in g of active material/100 g in Table 1 below:

TABLE 1

| Compositions | A1 (comparative) | A2 (invention) |
|---|---|---|
| Potassium persulfate | 10 | 10 |
| Ammonium persulfate | 10 | 10 |
| Potassium pyrophosphate | 10 | 10 |
| Hydroxyethylcellulose | 0.7 | 0.7 |
| Acrylate/C10-C30 alkyl acrylate | 0.7 | 0.7 |
| crosspolymer | | |
| Sodium lauryl sulfate | 1 | 1 |
| Liquid paraffin | 1.8 | 1.8 |
| Dimethicone | 1 | 1 |
| β-Cyclodextrin | 46.8 | 46.8 |
| Potassium carbonate | 18 | — |
| Ammonium carbonate | — | 18 |

Each of the compositions A1 and A2 is mixed with the composition B as described below: unless otherwise mentioned, the amounts are expressed in g of active material/100 g in Table 2 below:

TABLE 2

| Composition | B |
|---|---|
| Sodium salicylate | 0.04 |
| Acrylates/Beheneth-25 methacrylate copolymer | 0.4 |
| Tetrasodium etidronate | 0.06 |
| Tetrasodium pyrophosphate | 0.04 |
| Phosphoric acid | qs pH 3 |
| Cetearyl alcohol | 8 |
| Ceteareth-33 | 2 |
| Hydrogen peroxide | 9 |
| Water | q.s. 100 |

The lightening compositions are prepared by mixing:
1 part by weight of each of the compositions A1 and A2, and
1.5 parts by weight of composition B.

The pH of the compositions resulting from the mixing of the compositions, on the one hand, A1 and B, and on the other hand, A2 and B, is equal to 8.9. The mixtures obtained are applied to locks of natural pigmented Caucasian hair.

The “mixture/lock” bath ratio is, respectively, 10/1 (g/g).

The leave-on time is 50 min, on a hotplate set at 33° C. On conclusion of the leave-on time, the locks are rinsed and then dried in a ventilated oven at 60° C. for 20 minutes.

The colour of the locks was evaluated in the CIE L* a* b* system, using a Minolta CM3600D spectrocolorimeter (illuminant D65, angle 10°, specular components included).

The results obtained are collated in Table 3 below:

TABLE 3

| | L* | a* | b* | ΔE |
|---|---|---|---|---|
| Undyed lock | 20 | 2 | 2 | |
| Lock treated with composition | 64.5 | 8.2 | 26.6 | 51.2 |
| according to the invention | | | | |
| Lock treated with comparative | 50.2 | 11.7 | 30.4 | 42.6 |
| composition | | | | |

The mixture according to the invention derived from the composition A2 of the invention results in lightening performance qualities which are better than those observed for the comparative mixture outside the invention derived from the composition A1 not comprising any ammonium carbonate.

### Example 2

The compositions A3 and A4 as described below were prepared: unless otherwise mentioned, the amounts are expressed in g of active material/100 g in Table 4 below:

TABLE 4

| Compositions | A3 (comparative) | A4 (invention) |
|---|---|---|
| Potassium persulfate | 10 | 10 |
| Ammonium persulfate | 10 | 10 |
| Potassium pyrophosphate | 10 | 10 |
| Sodium silicate | 5 | 5 |
| β-Cyclodextrin | 50.5 | 50.5 |
| Sodium bicarbonate | 14.5 | — |
| Ammonium bicarbonate | — | 14.5 |

Each of the compositions A3 and A4 is mixed with the composition B as described above (cf. Table 2).

The pH of the compositions resulting from the mixing of the compositions, on the one hand, A3 and C, and on the other hand, A4 and C, is equal to 8.5.

The lightening compositions are prepared by mixing:
1 part by weight of each of the compositions A3 or A4, and
1.5 parts by weight of Composition B.

The composition resulting from the mixing of the composition A3 with the composition B is a comparative composition.

The composition resulting from the mixing of the composition A4 with the composition B is a composition according to the invention.

The mixtures obtained are applied to locks of natural pigmented Caucasian hair.

The “mixture/lock” bath ratio is, respectively, 10/1 (g/g).

The leave-on time is 50 min, on a hotplate set at 33° C. On conclusion of the leave-on time, the locks are rinsed and then dried in a ventilated oven at 60° C.

The colour of the locks was evaluated in the CIE L* a* b* system, using a Minolta CM 600D spectrocolorimeter (illuminant D65, angle 10°, specular components included).

The results obtained are collated in Table 5 below.

TABLE 5

| | L* | a* | b* | ΔE |
|---|---|---|---|---|
| Undyed lock | 20 | 2 | 2 | |
| Lock treated with composition | 65.3 | 7.3 | 26.3 | 51.7 |
| according to the invention | | | | |
| Lock treated with comparative | 61.0 | 7.7 | 24.6 | 47.2 |
| composition | | | | |

The mixture according to the invention derived from the composition A4 of the invention results in lightening performance qualities which are better than those observed for the comparative mixture outside the invention derived from the composition A3 not comprising any ammonium bicarbonate.

1. Composition for lightening keratin fibres, preferably human keratin fibres such as the hair, comprising:
a) one or more peroxygenated salts;
b) at least one agent chosen from ammonium carbonate, ammonium bicarbonate and a mixture thereof; and
c) at least 1% by weight of one or more polyphosphorus derivative(s) relative to the total weight of the composition, said polyphosphorus derivative(s) preferably being chosen from linear or cyclic compounds comprising at least two phosphorus atoms bonded together covalently via at least one linker L comprising at least one oxygen atom and/or at least one carbon atom,
said polyphosphorus derivative(s) being different from said peroxygenated salt(s).

2. Composition according to claim 1, characterized in that a) the peroxygenated salt(s) is/are chosen from persulfates, perborates, magnesium peroxide, and mixtures thereof, preferably from persulfates, perborates of alkali metals, such as potassium or sodium, magnesium peroxide and mixtures thereof, more preferentially from the persulfates, even more preferentially from alkali metal persulfates, calcium persulfates, ammonium persulfates and mixtures thereof, better still from potassium persulfates, ammonium persulfates and mixtures thereof.

3. Composition according to claim 1, characterized in that the total content of peroxygenated salt(s) ranges from 0.1% to 50% by weight, preferably from 1% to 40% by weight, more preferentially from 5% to 30% and better still from 10% to 25% by weight relative to the total weight of the composition.

4. Composition according to claim 1, characterized in that the total content of agent(s) b) ranges from 1% to 40% by weight, preferably from 5% to 40% by weight and more preferentially from 10% to 20% by weight relative to the total weight of the composition.

5. Composition according to claim 1, characterized in that said polyphosphorus derivative(s) comprises(comprise) at least two groups chosen from a group —P(R)(═O)—OH, a group —P(R)(═O)—OM, a group >P(═O)—OH and a group >P(═O)—O M, with:
M representing a cationic counterion, preferably chosen from alkali metals and alkaline-earth metals,
R representing a hydroxyl group, a group —OM′, M′ representing a cation counterion, preferably chosen from alkali metals and alkaline-earth metals, a (Ci-Ce)alkyl group, a (Ci-Ce)alkoxy group, a cycloalkyloxy group or a (hetero)aryloxy group, and
> representing the two bonds connected to the phosphorus atom and forming part of a ring.

6. Composition according to claim 1, characterized in that said polyphosphorus derivative(s) is/are chosen from compounds belonging to any one of formulae (I), (II) and (III) below, or mixtures thereof: 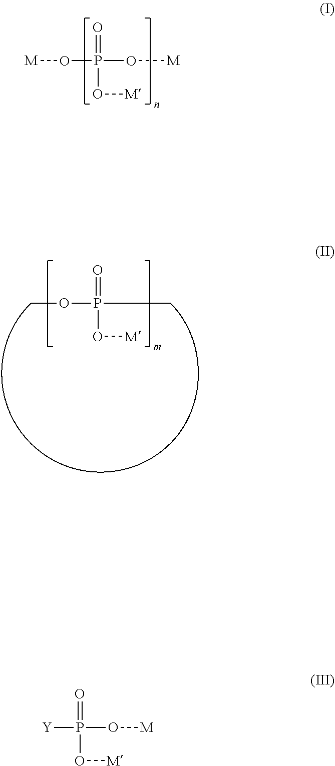
and also the solvates thereof such as hydrates,
in which formulae:
n ranges from 2 to 10, preferably from 2 to 6 and more preferentially from 2 to 3;
m ranges from 2 to 10, preferably from 2 to 6;
Y representing an alkyl chain comprising at least one phosphorus atom and optionally one or more non-phosphorus heteroatoms, or a cyclic carbon-based radical optionally comprising one or more heteroatoms, said hydrocarbon-based radical being substituted with one or more groups comprising one or more phosphorus atoms;
M or M′ representing, independently one another, a hydrogen atom, an alkali metal or an alkaline-earth metal;
representing a single bond when M or M¹ is H, or an ionic bond.

7. Composition according to claim 1, characterized in that the polyphosphorus derivative(s) is/are chosen from:
inorganic polyphosphorous derivatives chosen from:
pyrophosphates, preferably in the form of salts, preferably of alkali metal salts, which may or may not be hydrated, such as sodium pyrophosphate, potassium pyrophosphate or sodium pyrophosphate decahydrate;
hexametaphosphates, preferably in the form of salts, preferably of alkali metal salts, which may or may not be hydrated, such as sodium hexametaphosphate;
tripolyphosphates, preferably in the form of salts, preferably of alkali metal salts, which may or may not be hydrated, such as sodium tripolyphosphate;
trimetaphosphates, preferably in the form of salts, preferably of alkali metal salts, which may or may not be hydrated, such as sodium trimetaphosphate;
and mixtures thereof;
and organic polyphosphorus derivatives, preferably chosen from:
organic polyphosphate derivatives, such as polyphosphoric acids and/or salts thereof such as phytic acid;
organic polyphosphonate derivatives, such as polyphosphonic acids and/or salts thereof such as ethylenediaminetetramethylene phosphonic acid, diethylenetriaminepentamethylene phosphonic acid, aminotrimethylene phosphonic acid, hydroxyethylene diphosphonic acid, diethylenetriamine pentamethylene phosphonic acid and mixtures thereof;
and mixtures thereof.

8. Composition according to claim 1, characterized in that the polyphosphorus derivative(s) is/are chosen from:
inorganic polyphosphate derivatives chosen from hydrated or non-hydrated alkali metal pyrophosphates, such as sodium pyrophosphate, potassium pyrophosphate, sodium pyrophosphate decahydrate; and polyphosphates, such as sodium hexametaphosphate, sodium polyphosphate, sodium tripolyphosphate, sodium trimetaphosphate; and mixtures thereof;
organic polyphosphorus derivatives chosen from polyphosphoric acids and/or salts thereof, such as phytic acid, polyphosphonic acids and/or salts thereof such as ethylenediaminetetramethylene phosphonic acid, diethylenetriaminepentamethylene phosphonic acid, aminotrimethylene phosphonic acid, hydroxyethylene diphosphonic acid, diethylenetriamine pentamethylene phosphonic acid and mixtures thereof;
and mixtures thereof;
preferably from inorganic polyphosphate derivatives.

9. Composition according to claim 1, characterized in that the polyphosphorus derivative(s) is/are chosen from inorganic polyphosphate derivatives, preferably from hydrated or non-hydrated alkali metal pyrophosphates, more preferentially from sodium pyrophosphate, potassium pyrophosphate and sodium pyrophosphate decahydrate, and mixtures thereof.

10. Composition according to claim 1, characterized in that the total content of polyphosphorus derivative(s) c) ranges from 1% to 40% by weight, preferably from 2% to 30% by weight, more preferentially from 3% to 20% by weight and better still from 5% to 15% by weight relative to the total weight of the composition.

11. Composition according to claim 1, characterized in that it also comprises one or more thickeners, preferably chosen from organic thickeners, more preferentially from cellulose-based thickeners.

12. Composition according to claim 11, characterized in that the total content of thickener(s) ranges from 0.11% to 20% by weight and preferably from 0.1% to 10% by weight relative to the total weight of the composition.

13. Composition according to claim 1, characterized in that it comprises hydrogen peroxide.

14. Process for lightening keratin fibres, preferably human keratin fibres such as the hair, comprising the application to said keratin fibres of at least one composition as defined in claim 1.

15. Process according to claim 14, characterized in that it comprises a step of mixing a composition (A) with an oxidizing composition (B) comprising at least one chemical oxidizing agent, followed by the application to the keratin fibres of the composition resulting from the mixing of the compositions (A) and (B).

16. Process according to claim 15, characterized in that the composition resulting from the mixing of the composition (A) with the composition (B) is such that the total content of c) polyphosphorus derivative(s) is at least 1% by weight relative to the weight of the composition resulting from the mixing of the compositions (A) and (B).

17. Process according to claim 15, characterized in that the chemical oxidizing agent is hydrogen peroxide.

18. Device comprising at least two compartments, containing:
in a first compartment, a composition (A) as defined in claim 1;
in a second compartment, a composition (B) comprising at least one chemical oxidizing agent, preferably hydrogen peroxide,
the compositions (A) and (B) being intended to be mixed before application in order to obtain a composition resulting from the mixing of the compositions (A) and (B).
